(19) United States
(12) Patent Application Publication
(10) Pub. No.: US 2026/0114909 A1
(43) Pub. Date: Apr. 30, 2026

(54) EXPANDABL BONE IMPLANT FOR VETERINARY ORTHOPAEDIC SURGERY, ORTHOPAEDIC SYSTEM AND METHOD FOR MANUFACTURING THE IMPLANT

(71) Applicant: IN LIFE VET SA, Bulle (CH)

(72) Inventor: Guillaume LACAZE, La Rippe (CH)

(73) Assignee: IN LIFE VET SA, Bulle (CH)

(21) Appl. No.: 19/340,546

(22) Filed: Sep. 25, 2025

(30) Foreign Application Priority Data
Sep. 25, 2024 (EP) 24202390.1

(51) Int. Cl. A61B 17/70

(52) U.S. Cl. CPC A61B 17/7098; A61B 17/7071; A61B 17/7074; A61B 2503/40

(57) ABSTRACT

An expandable bone implant having folded and deployed configurations, two faces of the implant each including a flange for contact with bone tissue, each of the flanges having a central portion connected, by a hinge, to a pair of support arms, one arm of each pair connected by a hinge to the distal end while the other arm is connected by a hinge to the proximal end, movement of the distal and proximal ends towards one another causing the support arms to pivot, thus causing the flanges to move away from one another and, consequently, the expansion of the implant, wherein two other faces of the implant are covered in a fluidtight manner with one sheet per face, the sheet being a plastically deformable biocompatible metal alloy having a total surface area greater than or equal to the lateral surface area of the implant in the deployed configuration.

The present application relates to the field of surgery, in particular veterinary orthopaedic surgery and in particular to the treatment of a collapsed bone structure by restoring the volume of (or correcting) this bone structure. The present application relates in particular to an implant and to the method for manufacturing it, as well as to a system for restoring bone structure, in particular in the spine for the treatment (often called “reduction”) of compression fractures, in particular vertebral compression fractures (VCFs).

In this field, the problem of restoring the volume of bone structure that has collapsed is well known and the literature contains an abundance of solutions using expandable implants capable of passing from a folded configuration to a deployed configuration in order to restore the height of the bone structure, preferably in combination with an injection of bone substitute cement, known as cement (or bone cement). Many cements are known and they all have the advantage of being injectable in a liquid or viscous state for a certain period of time, then of hardening (by polymerization) inside the bone structure in order to stabilize it.

A major problem in this field relates to the expansion of the implant in order to restore height to the damaged bone tissue. Numerous solutions are known from the prior art, such as in particular from patent applications EP3086729, U.S. Pat. No. 11,540,926, EP3747385, EP2572680, EP3958752, EP2693967, EP2405835, U.S. Pat. No. 9,579,130, EP4216836, WO2023122005, WO2022162418, EP3843668 or U.S. Pat. No. 10,945,861 but these solutions present various problems in terms of difficulty of handling for deployment, and of stability and reliability once deployed. Moreover, these known solutions are generally accompanied by an injection of bone cement, but do not provide any teaching relating to cement leakage outside the implant, whereas such leakage can be detrimental to the surrounding tissues, or even to the entire organism if the chemical substances in the cement enter the bloodstream. In fact, cement generally comprises one or more polymerizable chemical substances, for example such as poly(methyl methacrylate) (PMMA) and possibly additives. In addition, the temperature reached during the polymerization of the cement is not harmless since it is generally greater than 60°.

Devices for correction and stabilization (or bone fracture reduction), particularly of the spine in the form of a stent, or in the form of porous inflatable bags or balloons, as in documents EP1408888 or EP1379185, possibly equipped with support flanges as in document US20060100706, are known from the prior art, in particular from documents EP1308134, U.S. Pat. Nos. 9,510,877, 8,936,627 or EP2467099. Numerous documents propose this type of stent, i.e., a deformable endoprosthesis similar to vascular stents, extenders or endoprostheses, which are generally in the form of a meshed tubular body, which is usually metallic and can be deformed by introducing an inflatable balloon in order to expand the body by spreading apart the meshes, with the balloon then being removed to allow cement to be injected, which hardens and thus forms a correcting and stabilizing structure. However, these devices have the drawback of requiring implantation in two stages: the inflation of the balloon and then the injection of cement, which slows down and complicates the operation and also presents a risk of the device collapsing between the deflation of the balloon and the filling of the stent with cement. Furthermore, these solutions have the disadvantage of not addressing the significant problem of cement leakage.

Solutions using implants with a mesh structure, made of shape memory metal, which is constrained into a folded shape for insertion into the bone tissue and capable of expanding spontaneously, when the stress is released and/or under the effect of heat, are also known, in particular from documents EP1938765, EP2351539 or WO200434924. These solutions have the disadvantage of using expensive alloys and involving complex manufacturing in order to obtain an adequate shape memory suitable for the intended implantation, which involves additional cost when multiplied by the number of different implants required to cover the various pathological cases, notably by the amplitude of the deformation the shape memory material can undergo. Moreover, the force exerted by the metal returning to its unstressed form is often not sufficient to properly correct the bone structure that has collapsed, or is at least a limiting factor. Furthermore, these solutions also have the disadvantage of not addressing the major problem of cement leakage.

Solutions using expandable implants that can be expanded using a lever mechanism, in the manner of a car jack, to restore the bone structure to a determined height are also known from the prior art, in particular from documents EP2405835, U.S. Pat. No. 9,579,130, EP2572680 or EP1956990. These solutions have the advantage of not risking collapse, unlike a stent that is deployed by a balloon that is removed before the cement is injected, but also have the drawback of implantation in two stages and of the fact that the dimensions of the support surface area for exerting the expansion force on the bone tissue are limited, in comparison with stents in particular.

Furthermore, they also have the drawbacks of being expensive and of likewise not addressing the major problem of cement leakage.

The problem of cement leakage has already been identified, in particular in documents EP1408888, EP1509175 or WO200394805, which explain the advantages that would be offered by a deformable implant that is not very permeable or that is impermeable, so as to limit or prevent cement leakage. These documents envisage numerous solutions for an expandable implant, made of metal or polymer, which could be either soft and flexible such as a membrane or a fabric, or even elastic, or semi-rigid (“conformable”) or rigid, or made of a shape memory material, with a continuous or fenestrated wall (i.e., mesh) and which could be porous or non-porous. However, all these hypotheses described in these documents define, above all, conceivable treatment methods and objectives to be achieved, without providing any real teaching as regards the technical features or the structural arrangement of the implants, or on how to obtain such implants and thus implement these methods. These proposals therefore present a major problem of technical feasibility.

Furthermore, one problem that is not identified in the prior art relates to the cement injection site and the distribution of the forces exerted on the bone tissues to correct them. Indeed, the impermeability of an implant makes it possible to avoid cement leakage, but the nature of the impermeable membrane and its technical features such as its physico-chemical and mechanical properties influence its ability to deploy without breaking and to correct the bone structure. Thus, an elastic membrane has the drawback of deforming excessively in low density regions and thus of having a limited ability to restore height, with, in addition, the risk of breaking at the points at which its maximum elasticity is exceeded because of this uncontrolled deformation. A semi-rigid membrane is therefore preferable, but this deformation problem also involves a problem in terms of shapes of the implant, in the folded configuration and especially in the deployed configuration. Specifically, the shape of the deployed implant delimits the cement injection site and the control of this site is important for the distribution of the forces leading to the filling of the low density regions while correcting the structure (especially in terms of height), and this has an impact on the success of the operation. Thus, it will be understood that the provision of an implant that addresses all of these problems is accompanied by a problem regarding technical feasibility and therefore manufacturing.

Other recurring problems in orthopaedic surgery relate to invasiveness (i.e. the aim of making the smallest possible incision and of minimizing lesions) and also to the deployment ratio in order to obtain a deployed implant that fills the largest possible volume while having been introduced through the smallest possible passage.

Furthermore, this deployment ratio will impact the distribution of forces for correcting the vertebrae: if the deformability is too great, the cement-injection pressure will deform the pouch rather than restoring the height.

A problem complementary to that of deployment relates to folding, which is generally not possible in implants of the prior art. Controlling folding allows the deployment to be controlled and therefore allows the injection site to be controlled with a uniform distribution of the cement and of the pressure to fill the space to be filled following the collapse. Perfect proportionality suited to the fracture while at the same time respecting the shape of the bone inside the fracture is thus achieved.

In this context, it will be understood that there is a technical problem that persists in this field relating to the restoration of bone structure (correction or reduction of fracture or increase in volume after collapse) by means of an expandable (deployable) implant that is able to expand collapsed bone tissue and is impermeable enough to prevent or limit the leakage of cement out of the implant with control of the injection site.

Finally, a main problem that persists in this field relates to the technical feasibility of manufacturing implants proposed in the prior art. Document WO200394805, for example, describes numerous methods of administering substances and in particular bone cement, with numerous variants envisaged for an expandable implant, made of metal or polymer, which could be either soft and flexible, such as a membrane or a fabric, or semi-conformable or rigid, or made of a shape memory material, with a continuous or fenestrated (i.e. meshed) wall and which could be porous or non-porous. However, that document describes only conceivable methods of treatment, but does not provide any teaching as to the technical features, or the structural arrangement, of these numerous hypothetical implants used for these envisaged methods, or on how to obtain such implants and thus actually implement these methods. These proposals therefore present a major problem of technical feasibility and define objects to be achieved rather than means for achieving them. Moreover, although many objectives have been detailed in the literature, many implants proposed for achieving these objectives have never seen the light of day due to manufacturing problems. In order to address the problem of manufacture, it is necessary to take into account the problems related to the desire to compact/fold a “bag” (balloon/pouch) made of a rigid and impermeable material in order to:
pass through a cylindrical conduit;
allow the pouch to expand without rupturing, despite the rigidity and the desired difference in volume between the folded volume and the deployed volume;
control the volume and the distribution of expansion forces on the bone.

In this context, one object of the present invention is to overcome at least certain drawbacks of the prior art by proposing an implant for restoring a collapsed bone structure that is reliable and simple to handle and to implant.

This purpose is achieved by an expandable bone implant for veterinary orthopaedic surgery for restoring the volume and/or geometry of a bone, by expansion between a folded configuration and a deployed configuration, said implant extending along a longitudinal axis between a proximal end that can cooperate with an implantation instrument for holding the implant and a distal end intended to be inserted first into the bone, at least two faces, for example an upper and a lower face, of the implant each comprising a flange for contact with bone tissue, each of the flanges comprising a central portion connected, by means of at least one hinge, to at least one pair of support arms each oriented in opposite directions within each pair, one arm of each pair being connected by a hinge to the distal end while the other arm is connected by a hinge to the proximal end, the implant being able to receive or comprising a central shaft extending through a sliding sleeve at the proximal end as far as a traction ring or socket at the distal end where it is able to transmit traction, when actuated by an instrument, to the distal end in order to allow it to move closer to the proximal end, causing the support arms to pivot, thus causing the flanges to move away from one another and, consequently, the expansion of the implant between the folded configuration and the deployed configuration, the implant being characterized in that:
at least two other faces of the implant, between those comprising the flanges, are covered with at least one sheet per face, said sheet being made of a biocompatible metal alloy and secured in a fluidtight manner to the central portions under the flanges, to lateral faces of the arms and to lateral faces of the proximal end and of the distal end,
said sheet is plastically deformable in order to allow the implant to expand and has, at least in the folded configuration, a plurality of antiform folds, referred to as convex, and synform folds, referred to as concave, said folds lying on top of one another in the folded configuration, the total surface area of said sheet being greater than or equal to the lateral surface area of the implant in the deployed configuration so as to form a fluidtight compartment that can receive a fluid inside the cavity obtained by the expansion of the implant.

According to another feature, the distance between a synform fold and the next antiform fold is longer than the distance between an antiform fold and the next synform fold, in order to make it easier to fold said sheet onto the surface of the lateral faces of the implant in the folded configuration.

According to another feature, said sheet of each lateral face of the implant is, in the deployed position, substantially diamond-shaped, with a permanent persistence of at least part of the folded folds near the proximal end and distal end.

According to another feature, said sheet is plastically deformable also from the folded configuration to the deployed configuration, in particular as a result of the persistence of the lying-down folds at the proximal and distal ends, facilitating the reversibility of the expansion.

According to another feature, said central shaft is able to cooperate with and/or is extended beyond the distal end of an implantation instrument at the proximal end of the implant and having an interior conduit in communication with a conduit formed inside said central shaft and opening onto the space formed by the parting of the flanges, through at least one opening allowing said fluid to be injected into the implant.

According to another feature, said sheet is secured to a lateral face of the proximal end by welding that fixes the proximal end of the lying-down and folded folds against the exterior wall of said sleeve and/or secured to a face of the distal end by welding that fixes the distal end of the lying-down and folded folds against the exterior wall of said socket.

According to another feature, the folds are, at least in the folded configuration, parallel to the longitudinal axis.

According to another feature that the number of folds is between 4 and 16, generally 6 to 12, and preferably of around 8.

According to another feature, the sheet has a thickness of between 3 and 100 microns, generally between 6 and 50 and preferably between 10 and 30 microns.

According to another feature, the sheet is made of a titanium alloy.

According to another feature, that the distance between the folds is variable from one lateral face of the implant to the other, so that the shape of the implant in the deployed configuration is asymmetric transversely to the longitudinal axis.

Another purpose of the present application is to overcome at least some of the disadvantages of the prior art by proposing a surgical intervention system that is easy to use and allows effective stabilization of bone tissue.

This purpose is achieved by a system for orthopaedic treatment of damaged bone tissue, comprising an implant, a bone substitute cement and at least one instrument for implantation and for injection of cement into said implant, said system being characterized the implant, characterized in that said implant is an implant.

According to another feature, that the instrument for implantation and for injection of cement comprises means for controlling the pressure and/or the aspiration of the cement so that the implant can be folded into the folded configuration if necessary.

According to another feature, the implantation instrument is different from but complementary to the injection instrument, the cement-injection canal of which passes through a canal inside a hollow rod of the implantation instrument, said hollow rod being configured to hold the proximal end of the implant at the distal end of said hollow rod.

Another purpose of the present application is to overcome at least some of the disadvantages of the prior art by proposing a method for manufacturing a surgical implant that is easy to use and allows effective stabilization of bone tissue.

This purpose is achieved by a method of manufacturing an implant according to the invention, said method being characterized in that it comprises:
a) Obtaining an expandable implant with two flanges that spread apart when the ends of the implant are brought together, thanks to support arms connecting these ends to the flanges;
b) Inserting a sheet of biocompatible metallic material between two racks equipped with triangular grooves to crease the sheet;
c) Folding the folds on top of each other to obtain a folded sheet;
d) Placing said folded sheet on one side of the implant;
e) Securing said sheet to said side;
f) Repeating steps b) to e) for the other side of the implant.

Other features and advantages of the present invention will become more clearly apparent on reading the following description of various embodiments, with reference to the appended drawings, in which:

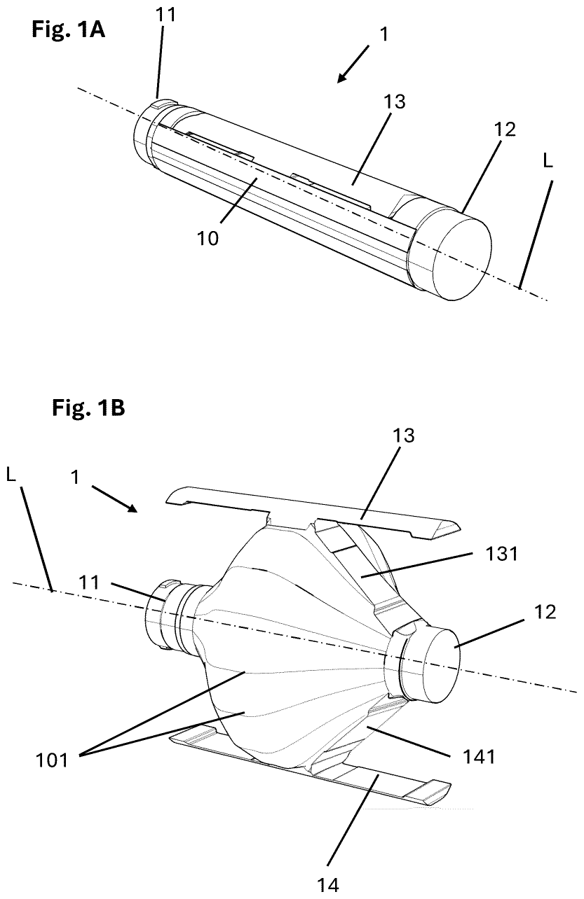

FIG. 1: FIG. 1A depicts a perspective view of an expandable implant in its folded configuration, according to certain embodiments, and FIG. 1B depicts a perspective view of this same implant in a deployed configuration;

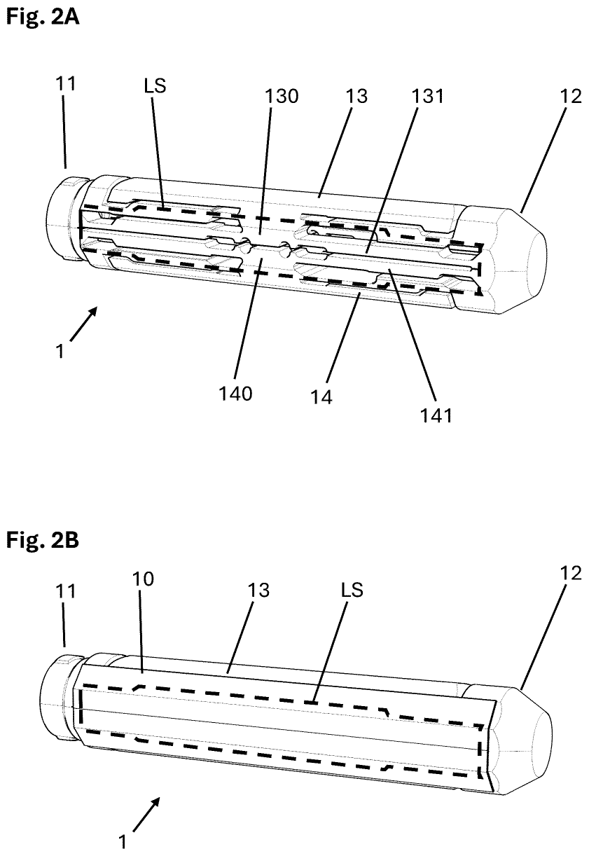

FIG. 2: FIG. 2A depicts a perspective view of an expandable implant from which the sheet has been removed and depicting the weld line of this sheet and FIG. 2B depicts the same implant in which the sheet is present and the weld line is depicted in dashed lines;

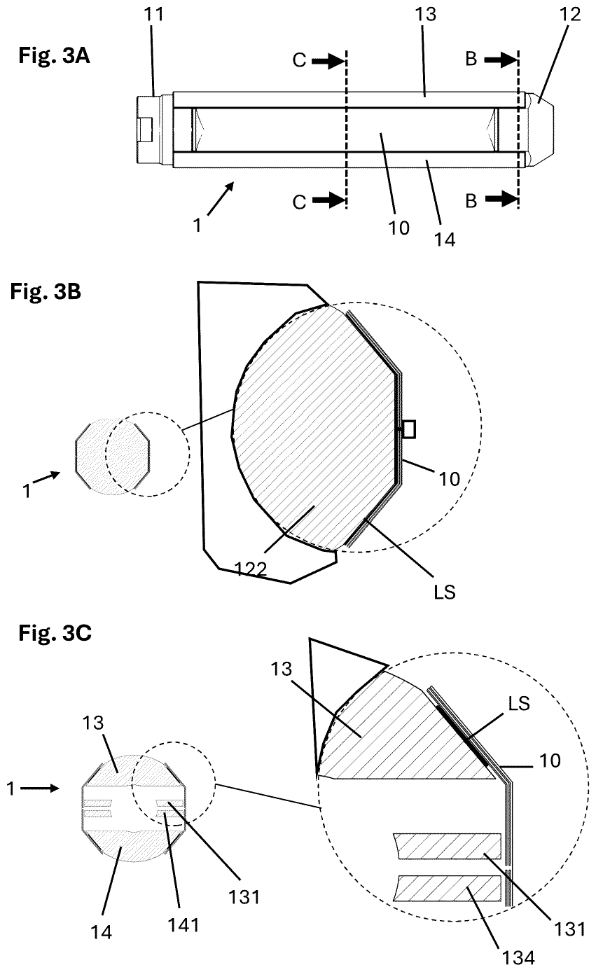

FIG. 3: FIG. 3A depicts a transparent view of an expandable implant according to certain embodiments, with the cross-sectional planes BB and CC of FIGS. 3B and 3C, respectively, that depict cross-sectional views, along the cross-sectional planes BB and CC, respectively, of FIG. A and each depict an enlargement showing the weld line of the sheet on the implant in these cross-sectional planes, according to certain embodiments;

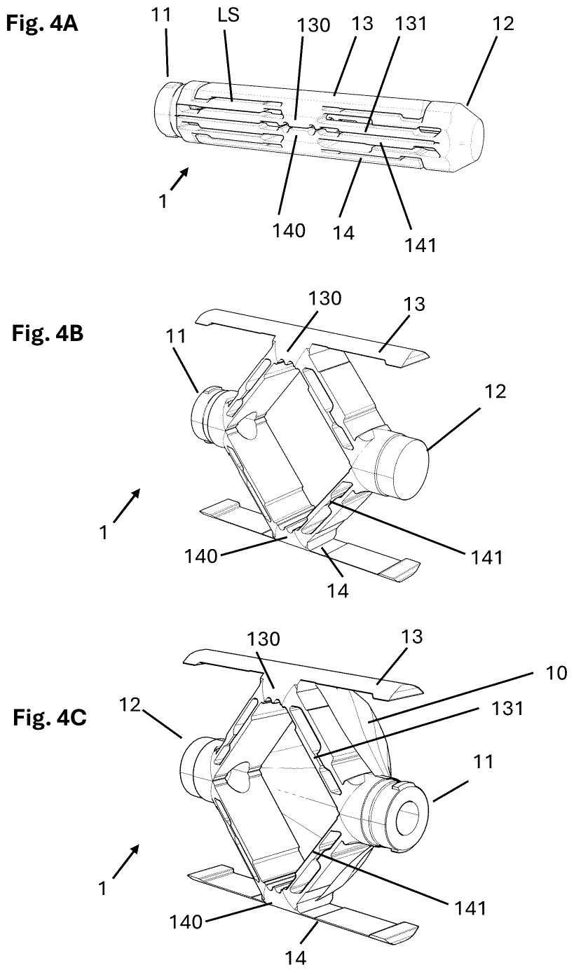

FIG. 4: FIG. 4A depicts a perspective view of an expandable implant in the folded configuration without its sheet, according to certain embodiments, and FIG. 4B depicts the same implant as in FIG. 4A but in the deployed configuration and FIG. 4C depicts an expandable implant fitted with a sheet on just one of its faces;

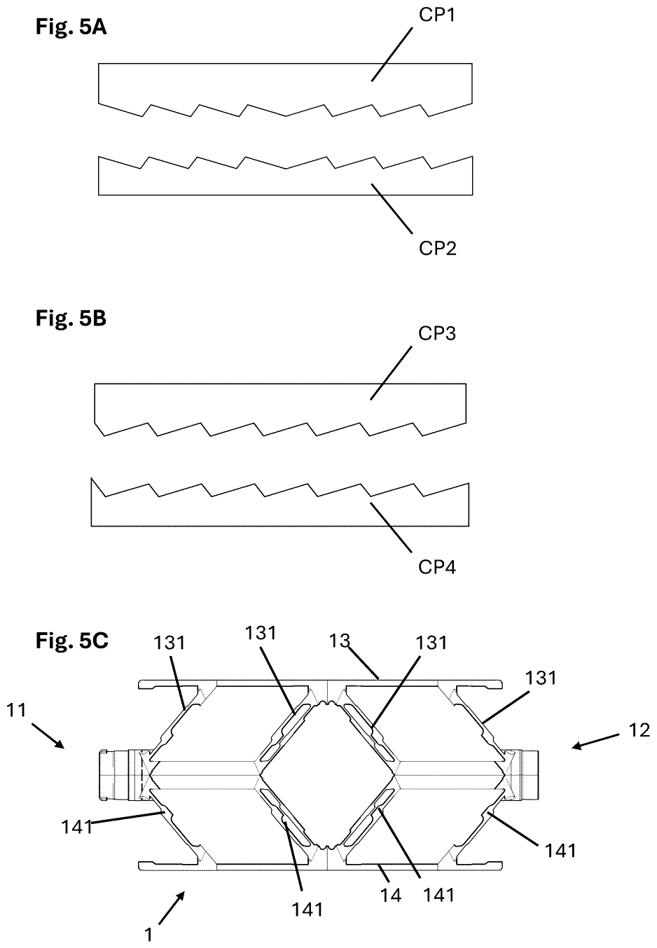

FIG. 5: FIG. 5A depicts a profile view of a rack for pre-folding sheets for expandable implants according to certain embodiments and FIG. 5B depicts a profile view of a pre-folding rack according to other embodiments and FIG. 5C depicts a profile view of an expandable implant with double support arms according to certain embodiments;

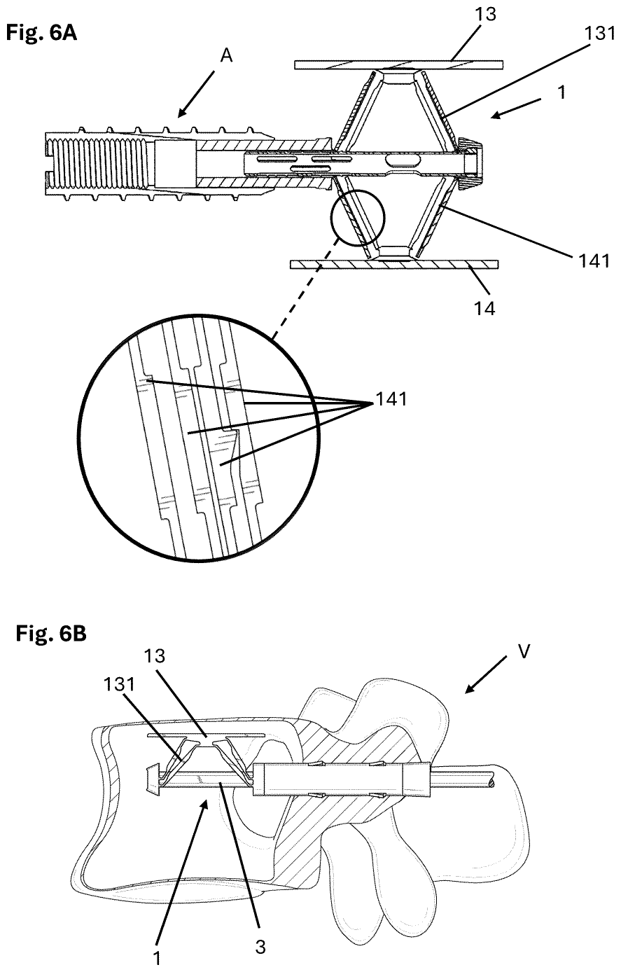

FIG. 6: FIG. 6A depicts a cross-sectional view of an expandable implant carried by an implantation instrument and with an enlargement showing details of the double support arms with a self-locking mechanism, FIG. 6B depicts a cross-sectional view of a vertebra in which an expandable implant according to other embodiments is implanted;

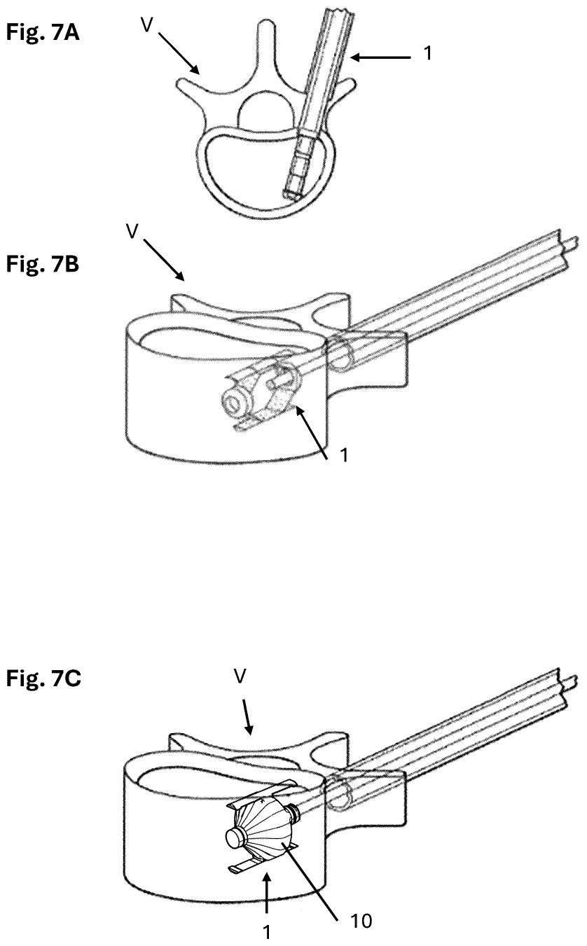

FIG. 7: FIG. 7A depicts a view from above of a vertebra in which an implant according to various embodiments is implanted, FIG. 7B depicts a perspective view of a vertebra in which an implant of the prior art is implanted, and FIG. 7C depicts a perspective view of a vertebra in which an implant according to certain embodiments is implanted;

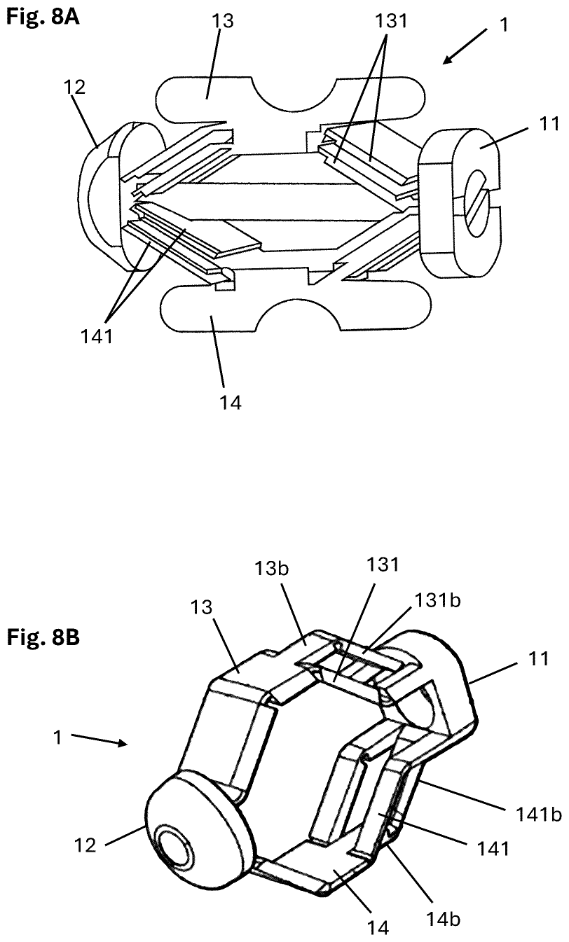

FIG. 8: FIG. 8A depicts a perspective view of an implant according to certain embodiments and FIG. 8B depicts a perspective view of an implant according to other embodiments;

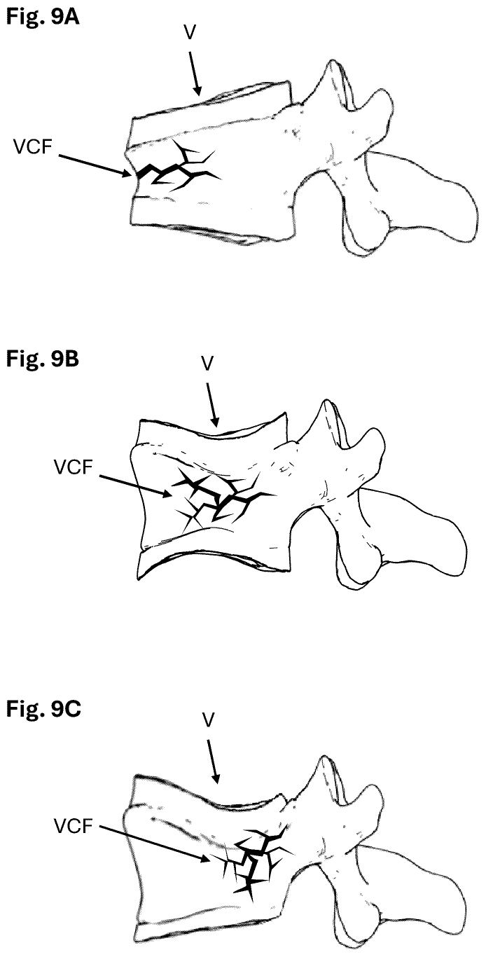

FIG. 9: FIGS. 9A, 9B and 9C depict profile views of vertebrae that have respectively suffered anterior, median and posterior vertebral compression fractures (VCFs);

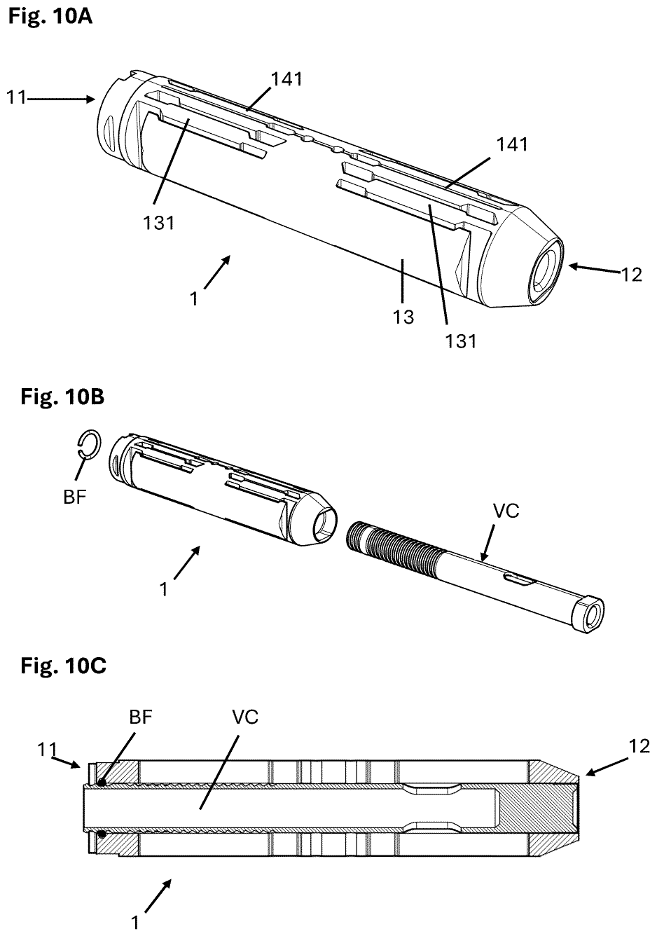

FIG. 10: FIG. 10A depicts a perspective view of an implant without its sheet according to certain embodiments and FIG. 10B depicts this same implant from which the expansion rod has been removed and FIG. 10C depicts a cross-sectional view of the implant of FIG. 10A in which the expansion rod is present.

The present application relates to an implant and to an orthopaedic surgery system for treating bone fractures and bone tissue in general, and to a method for manufacturing the implant. The bone implant is preferably a spinal implant, and in particular a vertebral or even in fact an intervertebral implant, although other uses can be contemplated elsewhere in the spine (intervertebral discs) or in other bone structures where a space left vacant as the result of a fracture needs to be filled (which can be the result of multiple causes, even though they generally involve a reduction in bone density). Thus, vertebral compression fractures (VCFs) are a preferred application but are not the only conditions that can be treated using the present invention, and a person skilled in the art will appreciate the possibilities that are offered without requiring further details herein. In terms of other bones, mention may be made of the femur or the humerus (humeral head), for example in the event of a risk of collapse. This application relates to an implant and a veterinary orthopedic surgery system for treating fractured bones and bone tissue in general, as well as a method for manufacturing the implant. The bone implant is preferably a spinal implant, and in particular a vertebral or even intravertebral implant, but other uses are possible elsewhere in the spine (intervertebral spines) or in other bone structures where it is necessary to fill a space left by a fracture (the causes of which may vary, although they generally involve a decrease in bone density). Thus, vertebral compression fractures (VCFs) are a favorite application, but they are not the only ones that can be treated with the present invention, and those skilled in the art will appreciate the possibilities offered without further detail here. Other bones include the femur or humerus (head), for example, in cases where there is a risk of collapse. In the veterinary field, it is known that animals sometimes have bone densities that are very different from those of humans and, above all, vary greatly depending on the species and even on the breed or animals within the same species, particularly in the case of dogs, whose physical properties vary enormously from one breed to another. For example, dachshunds and similar breeds have long (tall) vertebrae but are not very wide compared to other species. It is therefore useful to have expandable implants that allow for significant expansion in height while having a reduced length, and it is clearly necessary to take into account the differences in the shapes and sizes of the bones of different species in order to adapt the therapy effectively with suitable implants. In addition, some species, such as cats, have very rigid cortical bone but more flexible cancellous tissue than other species. It is therefore also necessary to take into account the nature of the bone tissue. Finally, another notable example concerns horses, which have bones, particularly vertebrae, with a specific anatomical shape and which sometimes bear a heavy load. Depending on the activity (e.g., sports) and morphology, bone density varies and implants must be adapted to allow for expansion but also to bear loads. Thus, for a horse, it may be necessary to have implants with more load-bearing arms (at least 3 or 4) than for other species (where 2 support arms are sometimes sufficient). In the absence of support arms, the implant must have sufficient mechanical strength, thanks to its ability to withstand higher cement pressure than in other cases.

In addition, the tibial plateau is frequently subject to crushing, and the implants or systems of the present application are useful for restoring height in any type of bone crushing or collapse, for example in the distal part of the humerus or femur. On the other hand, as taught, for example, in document EP2921142, it is possible to use expandable implants as bone anchoring implants, and such use is also possible for implants such as those of the present application. In this case, the implants will be extended at their proximal end by an elongated body to which another orthopedic implant of another type or a surgical device for fixing other elements can be attached.

Nevertheless, in the case of use as a bone anchor in a vascularized structure, such as a humeral or femoral head, the size of the implant will preferably be limited in relation to the bone structure in order to preserve vascularization and promote bone healing.

Certain embodiments provide for the injection of a fluid (e.g. “bone cement”, which is generally based on a polymer such as PMMA, for example, and is well known to those skilled in the art, and therefore no detail on the cement will be given here). Thus, once positioned, the implant can be stabilized by such an injection of cement. However, because cement leakages are a major problem in this field, various embodiments propose containing the cement in a fluidtight casing, the post-injection volume of which can be controlled by virtue of the structure and the material of the casing, as a function of the injected pressure (and the configuration of the bone tissue, preferably assessed in advance, as is the general practice in this field). Of course, fluidtightness is relative and this term is not limiting either, since the level of fluidtightness is actually adapted to the viscosity of the cement when it is injected. Some embodiments particularly allow proportional expansion of the casing by virtue of the (relative) flexibility of the sheet (10) of biocompatible metallic material. This material is generally a titanium alloy obtained in the form of a very thin sheet, preferably by rolling to give a controlled surface condition and a controlled thickness, notably a thickness comprised between 3 and 100 microns, generally between 6 and 50 and preferably 10 and 30 microns. Generally, the present invention uses at least one sheet (10) of biocompatible metal or biocompatible metal alloy, such as titanium or its alloys, particularly with nickel or others, but also nitinol or stainless steel or their alloys. Advantage is taken of recent techniques for obtaining very thin sheets of such metals, in particular with a thickness of less than 50 or even 40 um, which makes it possible to obtain relatively flexible and elastic sheets, but above all, which can be plastically deformed in a reversible manner without reaching their tear limit, by creating folds arranged longitudinally on the implant. In particular, it is possible to provide a maximum deployed volume that is greater than the volume required for the desired applications, so that this limit is never reached and it is possible to fold and then unfold the implant, even several times (for example, in the event of incorrect positioning of the implant). without risk of tearing and uncontrolled leakage. Thanks to this type of sheet and the configuration of their interlocking folds, it is possible to achieve expansion ratios between the folded volume and the deployed volume ranging from 2 to 20, or even 30, and it is also possible to control the shape of the implant in its deployed configuration, depending on the arrangement of the folds, in the same way as origami. Finally, although one of the main goals here is to prevent cement leakage, it may sometimes be useful to control the release of cement outside the implant, so that we no longer talk about leakage but rather controlled release, for example to allow adhesion to certain surrounding structures (usually bone structures). Similarly, since the injected fluid is not necessarily cement (or at least not the fluid that will come out of the implant), it may in fact be useful to administer molecules through such controlled release of this fluid. Thus, various embodiments provide for a certain porosity of the sheets (10) at least in certain portions of the implant, for example through holes of controlled microscopic size and controlled number and density. In any case, this sheet is capable of reversible plastic deformation for a number of times that is satisfactory for the target application, since it notably offers the possibility of retracting the casing formed by the sheet in the event of a problem (biocompatibility and resistance to tearing). Indeed, in general, controlling the metering of the cement allows the fifteen minutes of polymerization time to be monitoring, during which time it is possible to retract the casing and aspirate the cement. Furthermore, through the injection of cement and the expansion of the casing the implant fills the spaces in the damaged tissues as a function of the compression and bone-resistance forces relative to the hydraulic pressure supplied during the injection of cement. A closed structure needs to be obtained from such a sheet, which already means that the sheet needs to be closed on itself and locked in position. To this end, welding (or bonding or brazing, with these terms being non-limiting herein) can be used to join together two superposed edges or edges with interlocking turnups, in order to facilitate the welding and make it more robust. Some embodiments therefore contemplate closure by welding from the outside, which is simpler and more robust because of the superposition of layers in the vicinity of these complementary folds.

Various embodiments make it possible to obtain an expandable implant with very small dimensions in the folded configuration while at the same time guaranteeing a satisfactory volume in the deployed configuration. Thus, the passage required to introduce the implants of the present application is generally smaller than that of the known implants, whereas the expansion is greater than that of these known implants. Specifically, the folded volume or diameter is smaller than the deployed diameter by a factor of between 3 and 20, generally 3 to 8, preferably 4 to 7. This ratio depends of course on the amount of cement injected and certain embodiments take advantage of the fact that it is possible to provide an implant that is capable of deploying more than necessary, in particular by retaining folds in the deployed configuration. Thus, the volume of the implant will be determined based on the reduced size required for the introduction into the bone tissue and therefore with reference to the folded volume. However, different volumes are provided for the deployed configuration, since the number of folds and the length of the folds make it possible to increase the deployment ratio.

The term “secured” in this case means that two elements are secured to one another, either permanently (or quasi-permanently), but also sometimes means that a connection is made so that one element can be actuated by another. Thus, screw-fastening or collaboration between shapes for temporarily locking the elements together are covered by this nonlimiting term.

The terms ring, sleeve or tube refer to hollow structures such as bands, conduits or pipes, but in a non-limiting manner, in particular assuming various shapes (on the inside as on the outside), although a cylindrical shape is preferred. The term canal by contrast is preferably used herein to refer to a passage rather than to the element that contains it, and the term opening in this case refers to the fact that an element is open and able to be passed through, emerging into another structure or another element. In general, the terms sleeve, tube or conduit refer to longer elements than rings or bands, although their use herein likewise is non-limiting. Furthermore, the terms socket or cup also refer to hollow structures that are open at one end but are closed at the other end, such as plugs, closures, constrictions or restrictions, and these terms are used indiscriminately without any limitation.

The term “hinge” is used here in its functional sense without implying any structural limitation, and may in fact refer to mechanical hinges even though these are preferably formed (as illustrated in the non-limiting examples in the figures) by thinning (or narrowing, removing material from) elements such as the support arms or other elements. Thus, a hinge is in fact an articulation point or region, since it is known in this field that there is generally no danger associated with providing such pivot mechanisms for implants because the materials of which they are made are suited to this type of articulation.

The terms antiform fold, referred to as convex, and synform fold, referred to as concave, are used by analogy with the definitions of folds in numerous technical fields, including that of geology, but it will be appreciated that convexity is defined in this case with respect to the outside of the implant. An antiform or convex fold is therefore a fold that turns the material inwards, while an antiform fold turns the material outwards. The succession of the two types of folds makes it possible to limit as far as possible the volume that is folded. In addition, certain embodiments envisage a succession of long folds and of short folds making rolling and/or compaction easier by limiting the extent to which material is superposed in the folded configuration. It will also be noted that the number of folds is not limiting either but that it instead makes it possible to maintain the irregularity or trueness of shape of the implant as it deploys, this likewise offering advantages, in particular in terms of stabilization. In addition, it is still preferable to have an even distribution of surface areas between the folds for uniform deployment in order to enable uniform deployment, although the invention also envisages other applications and in particular folds of different sizes depending on the region of the implant, so as to obtain asymmetric deployment and better therapeutic outcomes. Moreover, the present invention makes it possible to control the shape of the implant once deployed by also setting the distance between the folds. Specifically, the distance between the synform/antiform folds, and therefore the distance between the long folds and the short folds, governs the way in which it deploys. Advantageously, if the density is greater at one point on the periphery, deployment will be greater and if it is lower, the casing will be able to deploy to a lesser extent. It will be appreciated that asymmetry and curving is thus obtained by more extensive deployment in regions that have the most folds. Likewise, it is possible to envisage more material (a larger surface area of the sheet) on one side, for example, so that the lateral expansion will be greater on this side than on the other. Moreover, in certain embodiments, the sheet is welded to the flanges and is therefore unable to deploy further than the distance between the flanges, which distance will have been set by the lever mechanism. What is thus obtained is an implant with limited expansion in one dimension (in general the essential dimension in which a precise height or width is desired to be restored), but not in another dimension, so that the injection of cement will deploy the casing into the volumes of low bone density that may be present around the implant. It will also be noted that the fluid injection instrument (Ac) may be fitted with means for controlling the injected pressure (a pressure gauge, for example) and for indicating the resulting volume, so that the expansion into the bone tissue may be controlled effectively. Finally, it will be appreciated that the instrumentation proposed in the present application in certain embodiments, using an implant holder (or ancillary) of relatively conventional type to hold the implant and introduce it into the bone tissue, but also of less-conventional type for expanding it into the bone tissue, also offers the advantage that all the implantation and stabilization steps can be carried out using a single instrument and in one continuous operation. Indeed, the ancillary with a hollow tube for conveying cement through the tube that holds the cement allows an instrument to be provided that allows the surgical intervention to be performed quickly and efficiently. After drilling, the implant is introduced and, without withdrawing the instrument, the casing can be inflated with the cement and then the tool can be withdrawn before, during or even after the polymerization of the cement (for example, using a mechanism for cutting the hardened cement as the instrument rotates). The time taken to perform the surgical operation is clearly markedly reduced, but also the stability of the implant is improved, which implant is not released at any time until it has been stabilised by the injection of cement filling all the free volumes around it, unlike in some solutions of the prior art.

The terms “cylinder”, “cylindrical” or “generalized cylinder” are used in the present application indiscriminately to make the invention easier to explain and in fact all refer to a “generalized cylinder”, i.e. a three-dimensional shape that is defined by a height (parallel to the longitudinal axis) and two bases (transverse to the longitudinal axis) that may have any shape, even though a circular shape is preferred in order to simplify manufacturing and limit the risk of lesion of the tissue into which it is introduced. Preferably, this “cylinder” is a right cylinder, i.e. its bases are aligned with respect to the generatrix (or height) of the cylinder. Furthermore, because the implant may be deployed in a tissue to conform to the shape of the space into which it has been introduced (modifying this by virtue of the pressure it exerts on this tissue), it is possible for the shape not to be constant and for the two bases of the cylinder to have different shapes (surface areas).

As a result, the term “diameter” is used, in the present application, to refer in fact to the longest dimension of the generalized cylinder transverse to the height (or longitudinal axis), i.e. in a plane (referred to as “transverse”) parallel to that of the bases of such a generalized cylinder. Thus, the term diameter may in fact refer to the length of the diagonal of a square or of a rectangle or else (for any shape) to the longest distance between two points included in such a transverse plane and lying on the circumference of such a cylinder. Likewise, the terms “circumference”, “periphery” or “perimeter” are used here to refer to the perimeter of these bases of any arbitrary shape.

Likewise, the terms “conical” or “frustoconical” are used here to denote shapes that widen from a minimum “diameter” (or area/surface area) up to a maximum “diameter”, but they do not imply any limitation as to the shape of the periphery, which may or may not be circular.

In general, the present application relates to an expandable bone implant (1) for veterinary orthopaedic surgery for restoring the volume and/or geometry of a bone, by expansion between a folded configuration and a deployed configuration, said implant extending along a longitudinal axis (L) between a proximal end (11) that can cooperate with an implantation instrument (A) for holding the implant and a distal end (12) intended to be inserted first into the bone, at least two faces, for example an upper and a lower face, of the implant each comprising a flange (13, 14) for contact with bone tissue, each of the flanges comprising a central portion (130, 140) connected, by means of at least one hinge, to at least one pair of support arms (131, 141) each oriented in opposite directions within each pair, one arm of each pair being connected by a hinge to the distal end (11) while the other arm is connected by a hinge to the proximal end (12), the implant (1) or comprising a central axis (3) or a housing able to receive such an axis extending through a sliding sleeve at the proximal end (11) as far as a traction ring or socket at the distal end (12) where it is able to transmit traction, when actuated by an instrument (A), to the distal end (12) in order to allow it to move closer to the proximal end (11), causing the support arms (131, 141) to pivot, thus causing the flanges (13, 14) to move away from one another and, consequently, the expansion of the implant between the folded configuration and the deployed configuration. This proximal end can be connected to a gripping instrument (known as an implant holder) and is therefore capable of cooperating with the latter by means of attachment or physical connection, for example, as known to those skilled in the art.

Nevertheless, certain embodiments provide specific and advantageous attachment means to facilitate the gripping of the implant by an implant holder and, above all, the release of the implant by an L-shaped movement of the implant holder. Furthermore, by being connectable to the instrument, the implant can generally be actuated for expansion (in particular by injecting a fluid into it and/or by pushing or pulling on an element of the implant), as is widely known in the prior art. Indeed, many systems comprise expandable implants that can be actuated when mounted on an implant holder that includes an actuating means for expanding the implant (generally a conduit and/or a rod passing through the implant holder to open into a cavity in the implant and/or cooperate with an implant component that allows its expansion, the actuation generally involving a pushing and/or pulling force). The skilled person will therefore understand from reading this application that the implant can be defined without further detail on the instrument and the actuation, since these are conventional mechanisms in the field and the system comprising the implant and the instrument is of course fully defined, but that the implant alone is in fact also well defined in its actuatable nature. mechanisms in the field and that the system comprising the implant and the instrument is of course fully defined, but that the implant alone is in fact also well defined in its operable nature independently of the instrument and without unnecessary detail on the operating mechanism (sliding rod, for example) insofar as these are perfectly standard or conventional mechanisms in the field. It is understood in the field of the present application that the term “operable” implies pushing or pulling (with or without rotation), and the present application thus provides sufficient explanation for the implant to be considered sufficiently clearly defined without additional reference to the instrument enabling its operation. Conversely, certain embodiments may relate to the instrument itself, through the originality of its elements allowing the implant to be grasped and/or actuated for expansion, and these characteristics then define the instrument independently of the implant, since they do not require any particular details about the implant other than those relating to the function performed by the instrument.

Such an implant (1) is preferably characterized in that
at least two other faces of the implant, between those comprising the flanges, are covered with at least one sheet (10) per face, said sheet being made of a biocompatible metal alloy and secured in a fluidtight manner to the central portions (130, 140) under the flanges, to lateral faces of the arms (131, 141) and to lateral faces of the proximal end (11) and of the distal end (12),
said sheet (10) is plastically deformable in order to allow the implant to expand and has, at least in the folded configuration, a plurality of antiform folds (101), referred to as convex, and synform folds (102), referred to as concave, said folds lying on top of one another in the folded configuration, the total surface area of said sheet being greater than or equal to the lateral surface area of the implant in the deployed configuration so as to form a fluidtight compartment that can receive a fluid inside the cavity obtained by the expansion of the implant.

In certain embodiments, the distance between a synform fold and the next antiform fold is longer than the distance between an antiform fold and the next synform fold, in order to make it easier to fold said sheet onto the surface of the lateral faces of the implant in the folded configuration.

In certain embodiments, said sheet of each lateral face of the implant is, in the deployed position, substantially diamond-shaped, with a permanent persistence of at least part of the folded folds near the proximal end (11) and distal end (12). In general, it is understood that the implant will retain, even in the deployed configuration, at least some of the folds of the sheet near the proximal and distal ends, but the dimensions and strength properties of the sheet (10) used allow the implant to be obtained and these persistent folds do not interfere with function or cause mechanical or physiological problems in the bone tissue.

In certain embodiments, said sheet is plastically deformable also from the folded configuration to the deployed configuration, in particular as a result of the persistence of the lying-down folds at the proximal and distal ends, facilitating the reversibility of the expansion.

In certain embodiments, said central shaft (3) is able to cooperate with and/or is extended beyond the distal end of an implantation instrument (A) at the proximal end of the implant and having an interior conduit in communication with a conduit (31) formed inside said central shaft (3) and opening onto the space formed by the parting of the flanges, through at least one opening (32) allowing said fluid to be injected into the implant (1). As is known, the instrument may include an expansion rod (A3) mounted freely in the instrument and allowing the implant to be activated by bringing the distal end closer to the proximal end and achieving expansion.

In certain embodiments, attached to a lateral face of the proximal end (11), with the proximal end of the flattened and folded folds welded against the outer wall of said sleeve and/or secured to a face of the distal end (12) with the distal end of the flattened and folded folds welded against the outer wall of said sleeve by a weld (120).

In certain embodiments, the folds are, at least in the folded configuration, parallel to the longitudinal axis (L).

In certain embodiments, the number of folds is between 4 and 16, generally 6 to 12, and preferably of around 8. However, three folds may sometimes suffice, particularly for small implants for small animals or small bones. However, the greater the number of folds, the less the material will deform, the less risk there will be of tearing, and the easier it will be to unfold. Thus, it is possible to plan for up to 20 folds, even for medium-sized animals, and when it comes to large animals such as horses, it is possible to allow for 25 to 30 folds, or even more.

In certain embodiments, the sheet (10) has a thickness of between 3 and 100 microns, generally between 6 and 50, and preferably 10 and 30, microns.

In certain embodiments, the sheet (10) is made of titanium alloy.

In certain embodiments, the distance between the folds is variable from one lateral face of the implant to the other, so that the shape of the implant in the deployed configuration is asymmetric transversely to the longitudinal axis (L).

In certain embodiments, at least part of the support arms is provided in duplicate. Specifically, in particular in order to limit the risks of non-regular expansion of the flanges, on account of the support arms in force opposition and the external stresses applied to the implant, it is preferable to provide support arms in duplicate, for example as depicted in FIGS. 6A, 6B, 8A and 8B.

In addition, such double arms may comprise a self-locking mechanism that locks them in the deployed configuration, such as, for example, teeth formed facing one another so that they engage with one another, for example as depicted in the enlarged box in FIG. 6A.

Furthermore, in order to further improve the reliability and symmetry of the expansion, it is possible to provide support arms that are offset towards the ends of the flanges instead of the single support arms that are articulated at the centre of the flanges, for example as depicted in FIG. 5C. Such a configuration makes it possible to create deformable parallelograms that retain their property of parallelism between their sides, thus making the expansion more reliable.

As regards actuation and locking, it should be noted that the very narrow diameters of the implants and their central shafts are not easily compatible with screw threads for expanding by means of a screwing action at the implant, although it is advantageous to perform a screwing action at the instrument actuating the expansion, in particular when the expansion involves bringing the support arms closer to one another. Thus, as is known from the prior art, it is possible to use, for example, a split ring that is housed in a circular recess of the implant and interacts with teeth on the push-or pull-shaft, said teeth being oriented in such a way as to allow this shaft to turn in just one direction, for example as depicted in FIGS. 10A, 10B and 10C. Thus, in order to expand the flanges, the shaft can be actuated by rotating past successive teeth, thereby making it possible to form a toothed lock (VC) for locking the implant in the deployed configuration.

The present application also relates to a system for orthopaedic treatment of damaged bone tissue, comprising a bone substitute cement and at least one instrument (A) for implantation and for injection of cement into the implant, characterized in that it comprises an implant according to one of the embodiments described here.

In certain embodiments, the instrument for implantation and for injection of cement comprises means for controlling the pressure and/or the aspiration of the cement so that the implant can be folded into the folded configuration if necessary.

In certain embodiments, the implantation instrument is distinct from but complementary to the injection instrument, the cement-injection canal of which passes through a canal inside a hollow rod of the implantation instrument, said hollow rod being configured to hold the proximal end of the implant at the distal end of said hollow rod.

In certain embodiments, the implant may comprise a second sheet (10), which surrounds the first sheet and is made from the same material or another material, for example to provide thermal insulation to protect the tissue from the heat of polymerization. In that case, the ends comprise an additional ring for fixing this second sheet in place, maintaining a space between it and the first sheet, potentially with an inlet for injecting another fluid between the two. During manufacture, these two sheets can then be folded and rolled at the same time.

These embodiments enable the injection site to be preformed (by compressing spongy bone tissue) enabling, for example, said fluid to be injected in two stages for better adjustment of the shape, of the resulting temperature in the tissue and/or of the rate of polymerization of the fluid.

The present application also relates to a method for manufacturing an implant according to one of the embodiments described here, characterized in that it comprises:
obtaining an expandable implant with two flanges that can be parted under the effect of the ends of the implant being moved closer together by support arms that connect these ends to the flanges;
inserting a sheet of biocompatible metal material between two racks equipped with grooves with a triangular profile in order to stamp folds into the sheet;
folding the folds on top of one another, in order to obtain a folded sheet;
arranging said folded sheet on one lateral face of the implant;
securing said sheet onto said lateral face;
repeating steps b) to e) for the other face of the implant.

For example, rolling may be achieved by introducing the closed and pre-folded sheet into a conduit with a diameter that narrows progressively down to the desired diameter for the implant, by sliding and twisting the implant in this conduit (for example with a guide inside the sheet to prevent it from becoming crushed).

The act of securing to the sleeve and the socket will generally be achieved using welding (120), preferably with the sheet being crushed beforehand onto the circumference of the socket or the sleeve, for example by means of a compression ring (121), examples of which are depicted in certain figures. Specifically, while it is possible to weld directly, compressing the folds in place remains preferable.

The illustrative and non-limiting figures of the present application will now be described in detail in order to better explain the various embodiments and to provide examples of structural elements that can be used within the context of the foregoing. That which follows must not be considered as being limiting since the various elements or components illustrated are merely examples and the figures may combine elements or components that are not necessarily dependent on one another.

FIG. 1A depicts a perspective view of an expandable implant in its folded configuration, according to certain embodiments, and FIG. 1B depicts a perspective view of this same implant in a deployed configuration. In FIG. 1B, it can be seen that the sheet (10) is welded to the arms 131-141 between the flanges and the latter are therefore free to move apart from one another and the sheet will conform to the shape of the implant during its deployment.

FIG. 2A depicts a perspective view of an expandable implant from which the sheet has been removed and depicting the weld line of this sheet while FIG. 2B depicts the same implant in which the sheet is present and the weld line is depicted in dashed lines. FIG. 2A depicts the path of welding of the sheet to the implant body and in particular to the central reinforcements and the arms of the implant and FIG. 2B also shows that the sheet can protrude beyond the welding since it is flexible and no additional adaptation is required.

FIG. 3A depicts a transparent view of an expandable implant according to certain embodiments, with the cross-sectional planes BB and CC of FIGS. 3B and 3C, respectively, that depict cross-sectional views, along the cross-sectional planes BB and CC, respectively, of Figure A and each depict an enlargement showing the weld line of the sheet on the implant in these cross-sectional planes, according to certain embodiments. These figures thus make it possible to show that, at the ends of the implant, the welding is continuous over an entire face of the implant, while at the deployable part of the implant, the welding is carried out, at the centre of the implant along the longitudinal axis, only on the central reinforcements (130, 140) and, between this centre and the proximal and distal ends, only on the support/deployment arms (131, 141) to allow the flanges to move apart and the sheet (10) welded to these reinforcements (130, 140) and arms (131, 141) to be deployeddeployed.

FIG. 4A depicts a perspective view of an expandable implant in the folded configuration without its sheet, according to certain embodiments, and FIG. 4B depicts the same implant as in FIG. 4A but in the deployed configuration and FIG. 4C depicts an expandable implant fitted with a sheet on just one of its faces. FIGS. 4A and 4B depict embodiments similar to the prior art in which the implant does not comprise a sheet for limiting cement leakage, but FIG. 4C depicts such an implant having just one face covered with the casing in order to show the advantage of being able to retain a determined volume of cement, it being possible for this determined volume to be variable from one face of the implant to the other depending on the number of folds produced.

FIG. 5A depicts a profile view of a rack for pre-folding sheets for expandable implants according to certain embodiments and FIG. 5B depicts a profile view of a pre-folding rack according to other embodiments and FIG. 5C depicts a profile view of an expandable implant with double support arms according to certain embodiments. Thus, FIGS. 5A and 5B depict two different variant embodiments for obtaining short folds and long folds, allowing folds to be folded on top of one another on each face of the implant and preserving the general shape of the implant (whereas identical symmetrical folds would involve the folds of the sheet being superposed in a folded configuration, generating an excess of material on the lateral faces of the implant). In the variant in FIG. 5A, the long folds and the short folds are formed symmetrically on either side of the median axis (height) of the implant while, in FIG. 5B, the distribution of the long folds and the short folds is continuous in the same direction over the entire face of the sheet and thus the folds obtained are all oriented in the same direction on each face of the implant to which such a sheet will be welded. FIGS. 5A and 5B depict folding tools in the form of racks defining a mean plane for compressing the sheet (10) and obtaining folds in this mean plane, but it is possible to provide alternatively (or in addition) at least one toothed wheel cooperating with a toothed rack or another toothed wheel to produce the same type of folding as the planar racks in FIGS. 5A and 5B.

Furthermore, FIG. 5C depicts a variant embodiment in which the number of deployment arms has been doubled by adding additional arms near to the ends of the flanges. This type of configuration provides better stability and better reliability during deployment since the presence of pairs of arms on each side of the implant with respect to its centre, along the longitudinal axis, provides a configuration comprising deformable parallelograms that limit the risks of non-parallel spacing of the flanges while, when single arms are present, there is a general risk of the flanges being undesirably deployed non-parallel to one another on account of the resistive forces that the flanges encounter against the surrounding tissue during the expansion of the implant.

FIG. 6A depicts a cross-sectional view of an expandable implant carried by an implantation instrument and with an enlargement showing details of the double support arms with a self-locking mechanism, FIG. 6B depicts a cross-sectional view of a vertebra in which an expandable implant according to other embodiments is implanted. FIG. 6A depicts a partially cross-sectional profile view of an implant of the prior art in which the deployment arms comprise self-locking structures enabling the implant to be locked in the deployed position by virtue of the cooperation of teeth that interlock when the implant is completely deployed. This type of structure known from the prior art can of course also be used in the present invention. FIG. 6B depicts a variant of the prior art in which the implant also has just one flange and therefore allows deployment in a single given direction perpendicular to the longitudinal axis, which may be useful in certain cases. The present invention of course also makes it possible to carry out this type of embodiment.

FIG. 7A depicts a view from above of a vertebra in which an implant according to various embodiments is implanted, FIG. 7B depicts a perspective view of a vertebra in which an implant of the prior art is implanted, and FIG. 7C depicts a perspective view of a vertebra in which an implant according to certain embodiments is implanted.

FIG. 8A depicts a perspective view of an implant according to certain embodiments and FIG. 8B depicts a perspective view of an implant according to other embodiments. FIG. 8A depicts a variant embodiment in which the flanges have a particular shape in order to conform to the shape of bone structures to be treated with the implant, such as for example vertebral discs, to form an interspinous implant. Of course, the present invention also makes it possible to produce this type of flange shape as well as any other shape for geometrical restoration of various bone structures of various shapes, whether they are vertebral or not. FIG. 8B depicts another variant embodiment known from the prior art, in which the deployment arms (131, 141), at least on the side of one of the ends of the implant, are supported by a second pair of arms (131b, 141b) providing improved stability by creating a deformable parallelogram that improves reliability of the deployment of the flanges. However, this embodiment may prove to be less effective than the one detailed above in FIG. 5C, which has parallelograms that are more effective because they are to a greater extent spaced apart proportionally with respect to the size of the implant.

FIGS. 9A, 9B and 9C depict profile views of vertebrae that have respectively suffered anterior, median and posterior vertebral compression fractures (VCFs). The invention allows this kind of vertebral fracture to be treated by arranging the deployable implant in the correct position in the plane of the implantation site, using antero-posterior and/or medio-lateral positioning and by adjusting the depth of insertion and/or the angle of insertion of the implant, according to the type of surgical approach being used (for example lateral, anterior, dorsal, transforaminal, transpedicular, etc.).

FIG. 10A depicts a perspective view of an implant without its sheet according to certain embodiments and FIG. 10B depicts this same implant from which the expansion rod has been removed and FIG. 10C depicts a cross-sectional view of the implant of FIG. 10A in which the expansion rod is present. FIG. 10A depicts an embodiment in which deployment is achieved by a mechanism known from the prior art. As illustrated in FIG. 10B, this mechanism comprises a toothed lock (VC) containing a circumferential groove that can receive a split ring acting as a locking clip intended to cooperate with a circumferential housing inside the conduit into which the toothed lock is inserted to form this clip. As depicted in FIG. 10C, as the toothed lock is pulled out, the split ring cooperates with teeth in the conduit of the implant, which can thus be locked incrementally by this lock pressing against interior teeth in the conduit of the implant. These teeth are preferably asymmetrical so as to allow the lock to be withdrawn only towards the outlet, thereby keeping the implant secure in the deployed position and preventing it from re-folding under the force exerted by the surrounding tissue.

The present application describes various technical and advantageous features with reference to the figures and/or to various embodiments. A person skilled in the art will appreciate that the technical features of one given embodiment may in fact be combined with features of another embodiment unless explicitly stated otherwise or unless it is obvious that these features are incompatible or that combining them will not provide a solution to at least one of the technical problems mentioned in the present application. In addition, the technical features described in one given embodiment may be taken in isolation from the other features of this embodiment unless explicitly stated otherwise.

### DETAILED LIST OF REFERENCES IN THE FIGURES

1 implant
10 sheet
101 fold
11 proximal end
LS weld line
12 distal end
A implantation instrument
CP1 first pre-folding rack
CP2 second pre-folding rack
CP3 third pre-folding rack
CP4 fourth pre-folding rack
3 central shaft
31 conduit in the central shaft
32 openings of the conduit of the central shaft
13 first flange
14 second flange
15 third flange
130 central portion of the first flange
140 central portion of the second flange
131 support arm of the first flange
141 support arm of the second flange
VC toothed lock

1. An expandable bone implant (1) for veterinary orthopaedic surgery for restoring the volume and/or geometry of a bone, by expansion between a folded configuration and a deployed configuration, said implant extending along a longitudinal axis (L) between a proximal end (11) connectable with an implantation instrument (A) for holding the implant and a distal end (12) intended to be inserted first into the bone, at least two faces, for example an upper and a lower face, of the implant each comprising a flange (13, 14) for contact with bone tissue, each of the flanges comprising a central portion (130, 140) connected, by means of at least one hinge, to at least one pair of support arms (131, 141) each oriented in opposite directions within each pair, one arm of each pair being connected by a hinge to the distal end (11) while the other arm is connected by a hinge to the proximal end (12), the implant (1) being able to receive or comprising a central shaft (3) extending through a sliding sleeve at the proximal end (11) as far as a traction ring or socket at the distal end (12) where it is able to transmit traction, when actuated by an instrument (A), to the distal end (12) in order to allow it to move closer to the proximal end (11), causing the support arms (131, 141) to pivot, thus causing the flanges (13, 14) to move away from one another and, consequently, the expansion of the implant between the folded configuration and the deployed configuration,
wherein:
at least two other faces of the implant, between those comprising the flanges, are covered with at least one sheet (10) per face, said sheet being made of a biocompatible metal alloy and secured in a fluidtight manner to the central portions (130, 140) under the flanges, to lateral faces of the arms (131, 141) and to lateral faces of the proximal end (11) and of the distal end (12),
said sheet (10) is plastically deformable in order to allow the implant to expand and has, at least in the folded configuration, a plurality of antiform folds (101), referred to as convex, and synform folds (102), referred to as concave, said folds lying on top of one another in the folded configuration, the total surface area of said sheet being greater than or equal to the lateral surface area of the implant in the deployed configuration so as to form a fluidtight compartment that can receive a fluid inside the cavity obtained by the expansion of the implant.

2. The expandable bone implant according to claim 1, wherein the distance between a synform fold and the next antiform fold is longer than the distance between an antiform fold and the next synform fold, in order to make it easier to fold said sheet onto the surface of the lateral faces of the implant in the folded configuration.

3. The expandable bone implant according to claim 1, wherein said sheet of each lateral face of the implant is, in the deployed position, substantially diamond-shaped, with a permanent persistence of at least part of the folded folds near the proximal end (11) and distal end (12).

4. The expandable bone implant according to claim 1, wherein said sheet is plastically deformable also from the folded configuration to the deployed configuration, in particular as a result of the persistence of the lying-down folds at the proximal and distal ends, facilitating the reversibility of the expansion.

5. The expandable bone implant according to claim 1, wherein said central shaft (3) is able to cooperate with and/or is extended beyond the distal end of an implantation instrument (A) at the proximal end of the implant, the said instrument having an interior conduit in communication with a conduit (31) formed inside said central shaft (3) and opening onto the space formed by the parting of the flanges, through at least one opening (32) allowing said fluid to be injected into the implant (1).

6. The expandable bone implant according to claim 1, wherein said sheet (10) is secured to a lateral face of the proximal end (11) with the proximal end of the lying-down and folded folds welded against the exterior wall of said sleeve by welding (110) and/or secured to a face of the distal end (12) by welding (120) that fixes the distal end of the lying-down and folded folds welded against the exterior wall of said socket by welding (120).

7. The expandable bone implant according to claim 1, wherein the folds are, at least in the folded configuration, parallel to the longitudinal axis (L).

8. The expandable bone implant according to claim 1, wherein the number of folds is between 4 and 16, generally 6 to 12, and preferably of around 8.

9. The expandable bone implant according to claim 1, wherein the sheet (10) has a thickness of between 3 and 100 microns, generally between 6 and 50, and preferably 10 and 30, microns.

10. The expandable bone implant according to claim of wherein the sheet (10) is made of titanium alloy.

11. The expandable bone implant according to claim 1, wherein the distance between the folds is variable from one lateral face of the implant to the other, so that the shape of the implant in the deployed configuration is asymmetric transversely to the longitudinal axis (L).

12. A system for orthopaedic treatment of damaged bone tissue, comprising an implant, a bone substitute cement and at least one instrument (A) for implantation and for injection of cement into said implant, said system being characterized in that said implant is an implant according to claim 1.

13. The system according to claim 12, wherein the instrument for implantation and for injection of cement comprises means for controlling the pressure and/or the aspiration of the cement so that the implant can be folded into the folded configuration if necessary.

14. The system according to claim 12, wherein the implantation instrument is different from but complementary to the injection instrument, the cement-injection canal of which passes through a canal inside a hollow rod of the implantation instrument, said hollow rod being configured to hold the proximal end of the implant at the distal end of said hollow rod.

15. A method for manufacturing an implant according to claim 1, said method comprising:
a) obtaining an expandable implant with two flanges that can be parted under the effect of the ends of the implant being moved closer together by support arms that connect these ends to the flanges;
b) inserting a sheet of biocompatible metal material between two racks equipped with grooves with a triangular profile in order to stamp folds into the sheet;
c) folding the folds on top of one another, in order to obtain a folded sheet;
d) arranging said folded sheet on one lateral face of the implant;
e) securing said sheet onto said lateral face;
f) repeating steps b) to e) for the other face of the implant.
